(19) United States
(12) Patent Application Publication
(10) Pub. No.: US 2006/0241717 A1
(43) Pub. Date: Oct. 26, 2006

(54) Treatment of movement disorders by extra dural motor cortex stimulation

(76) Inventors: Todd K. Whitehurst, Frazier Park, CA (US); James P. McGivern, Stevenson Ranch, CA (US)

Correspondence Address: ADVANCED BIONICS CORPORATION, 25129 RYE CANYON ROAD, VALENCIA, CA, 91355, US

(21) Appl. No.: 10/224,021

(22) Filed: Aug. 19, 2002

Related U.S. Application Data
(60) Provisional application No. 60/316,225, filed on Aug. 30, 2001.

(51) Int. Cl. A61N 1/36

(52) U.S. Cl. 607/45; 607/48

(57) ABSTRACT

System and methods for introducing one or more stimulating drugs and/or applying electrical stimulation to the cortex of the brain to treat movement disorders uses at least one implantable system control unit (SCU), specifically an implantable signal/pulse generator (IPG) or microstimulator with two or more electrodes in the case of electrical stimulation, and an implantable pump with one or more infusion outlets in the case of drug infusion. In certain embodiments, a single SCU provides both electrical stimulation and one or more stimulating drugs. In some embodiments, one or more sensed conditions are used to adjust stimulation parameters.

The present application claims the benefit of U.S. Provisional Patent Application Ser. No. 60/316,225, filed Aug. 30, 2001, which application is incorporated herein by reference in its entirety.

### FIELD OF THE INVENTION

The present invention generally relates to implantable drug delivery and electrical stimulation systems and methods, and more particularly relates to utilizing one or more implantable devices to deliver electrical stimulation and/or one or more stimulating drugs to the motor cortex as a treatment for movement disorders.

### BACKGROUND OF THE INVENTION

Movement disorders are neurologic syndromes characterized by either an excess or a paucity of movement. These disorders affect approximately two million Americans, including over one million suffering from benign essential tremor, and half a million suffering from Parkinson's Disease. A substantial percentage of those afflicted with movement disorders experience a significant decrease in quality of life, suffering such problems as incapacitating tremor, limited mobility, bradykinesia (difficulty consciously initiating movement), dysarthria (difficulty with speech), and consequent social isolation. The etiology of many movement disorders, e.g., benign essential tremor, is poorly understood. For other movement disorders, e.g., Parkinson's disease, the mechanism of the disorder and even the brain cells affected have been identified, but even with optimal medication and physician care the disease may not be reversed and may even continue to progress. Medications that are effective for movement disorders may have significant side effects and may lose their efficacy over time.

Parkinson's Disease is caused by a gradual loss of dopaminergic (i.e., dopamine-secreting) neurons in the substantia nigra. Consequently, levels of dopamine decrease in the striatum (i.e., the putamen and the caudate nucleus). Although dopamine has both excitatory and inhibitory effects on the striatum, the predominant effect of the loss of dopamine is decreased inhibition (by GABA) of the internal segment of the globus pallidus. This leads to increased GABA output from the internal segment of the globus pallidus, which inhibits the ventrolateral thalamus. This leads in turn to decreased inhibition of (and ultimately decreased control over) the motor cortex. The subthalamic nucleus appears to increase its activity in Parkinson's Disease as well, and this is believed to contribute to the symptoms of the disease.

Essential Tremor (ET), a.k.a., Benign Essential Tremor, is the most common movement disorder. It is a syndrome characterized by a slowly progressive postural and/or kinetic tremor, usually affecting both upper extremities. The prevalence of ET in the US is estimated at 0.3-5.6% of the general population. A 45-year study of ET in Rochester, Minn. reported an age- and gender-adjusted prevalence of 305.6 per 100,000 and an incidence of incidence of 23.7 per 100,000.

ET affects both sexes equally. The prevalence of ET increases with age. There are bimodal peaks of onset—one in late adolescence to early adulthood and a second peak in older adulthood. The mean age at presentation is 35-45 years. ET usually presents by 65 years of age and virtually always by 70 years. Tremor amplitude slowly increases over time. Tremor frequency decreases with increasing age. An 8-12 Hz tremor is seen in young adults and a 6-8 Hz tremor is seen in the elderly. Although ET is progressive, no association has been found between age of onset and severity of disability.

Mortality rates are not increased in ET. However, disability from ET is common. Significant changes in livelihood and socializing are reported by 85% of individuals with ET, and 15% report being seriously disabled due to ET. Decreased quality of life results from both loss of function and embarrassment. In a study of hereditary ET, 60% did not seek employment; 25% changed jobs or took early retirement; 65% did not dine out; 30% did not attend parties, shop alone, partake of a favorite hobby or sport, or use public transportation; and 20% stopped driving.

Additional and improved treatment options are needed for patients suffering from movement disorders.

### BRIEF SUMMARY OF THE INVENTION

The invention disclosed and claimed herein provides systems and methods for introducing one or more stimulating drugs and/or applying electrical stimulation to the extradural motor cortex for treating or preventing movement disorders, as well as the symptoms and pathological consequences thereof. According to some embodiments of the invention, the stimulation increases excitement of an area(s) of the brain, and specifically the portions of the cerebral cortex and/or other areas of the brain affected by stimulation to those portions of the cortex, thereby treating or preventing movement disorders. According to other embodiments of the invention, the stimulation decreases excitement of an area(s) of the brain, and specifically the portions of the cerebral cortex and/or other areas of the brain affected by stimulation to those portions of the cortex, thereby treating or preventing movement disorders.

The treatment provided by the invention may be carried out by one or more system control units (SCUs). In some forms of an SCU, one or more electrodes are surgically implanted to provide electrical stimulation from an implantable signal/pulse generator (IPG) and/or one or more infusion outlets and/or catheters are surgically implanted to infuse drug(s) from an implantable pump. When necessary and/or desired, an SCU may provide both electrical stimulation and one or more stimulating drugs. In other forms of an SCU, a miniature implantable neurostimulator (a.k.a., a microstimulator), such as a Bionic Neuron (also referred to as a BION® microstimulator) or the like, is implanted. For instance, a BION SCU(s) may be implanted substantially or entirely in the skull and/or in the extradural area under the skull or, alternatively, within the skull or even subcutaneously above the skull, with at least part in contact with the underlying dura. The systems of the invention may also include one or more sensors for sensing symptoms or other conditions that may indicate a needed treatment.

In some configurations, the SCU is implanted in a surgically-created shallow depression or opening in the skull, such as in the temporal, parietal, or frontal bone. In some such configurations, one or more electrode leads and/or catheters attached to the SCU run subcutaneously to an opening in the skull and pass through the opening into or onto the extradural area under the skull. The electrodes used for electrical stimulation may be arranged as an array on a very thin implantable lead, such as a paddle-shaped lead. The SCUs programmed to produce electrical stimulation may provide either monopolar electrical stimulation, e.g., using the SCU case as an indifferent electrode, or to produce bipolar electrical stimulation, e.g., using one of the electrodes of an electrode array as an indifferent electrode.

The SCU used with the present invention possesses one or more of the following properties, among other properties:
at least two electrodes for applying stimulating current to surrounding tissue and/or a pump and at least one outlet for delivering a drug or drugs to surrounding tissue;
electronic and/or mechanical components encapsulated in a hermetic package made from biocompatible material(s);
an electrical coil or other means of receiving energy and/or information inside the package, which receives power and/or data by inductive or radio-frequency (RF) coupling to a transmitting coil placed outside the body, thus avoiding the need for electrical leads to connect devices to a central implanted or external controller;
means for receiving and/or transmitting signals via telemetry;
means for receiving and/or storing electrical power within the SCU; and
a form factor making the SCU implantable in a depression or opening in the skull, and/or in the extradural area under the skull.

An SCU may operate independently, or in a coordinated manner with other implanted SCUs, other implanted devices, and/or with devices external to a patient's body. For instance, an SCU may incorporate means for sensing a patient's condition. Sensed information may be used to control the electrical and/or drug stimulation parameters of the SCU in a closed loop manner. The sensing and stimulating means may be incorporated into a single SCU, or a sensing means may communicate sensed information to at least one SCU with stimulating means.

### BRIEF DESCRIPTION OF THE DRAWINGS

The above and other aspects of the present invention will be more apparent from the following more particular description thereof, presented in conjunction with the following drawings wherein:

FIG. 1 is a lateral view of the cerebrum;

FIG. 2 illustrates a lateral view of the skull and components of some embodiments of the invention;

FIG. 3 illustrates internal and external components of certain embodiments of the invention;

FIGS. 4A, 4B, and 4C show possible configurations of an implantable microstimulator of the present invention;

FIG. 5 illustrates external components of various embodiments of the invention; and

FIG. 6 depicts a system of implantable devices that communicate with each other and/or with external control/programming devices.

Corresponding reference characters indicate corresponding components throughout the several views of the drawings.

### DETAILED DESCRIPTION OF THE INVENTION

The following description is of the best mode presently contemplated for carrying out the invention. This description is not to be taken in a limiting sense, but is made merely for the purpose of describing the general principles of the invention. The scope of the invention should be determined with reference to the claims.

Patients suffering from tremor (e.g., due to essential tremor or Parkinson's disease) and other symptoms may undergo surgery to lesion a part of the brain (e.g., the ventral intermediate (Vim) nucleus of the thalamus), which may afford some relief. Patients suffering from Parkinson's disease may undergo surgery to lesion another part of the brain (e.g., internal globus pallidus (Gpi), subthalamic nucleus (STN)). However, lesions are irreversible, and may lead to side effects such as dysarthria or cognitive disturbances. Additionally, lesions generally yield effects on only one side of the body (the contra-lateral side), and bilateral lesions are significantly more likely to produce side effects. Other surgical procedures, such as fetal tissue transplants, are costly and unproven.

High frequency chronic electrical stimulation (i.e., frequencies above about 100 Hz) of certain areas of the brain has been demonstrated to be as efficacious as producing a lesion in any one of those areas. In contrast to ablation surgery, chronic electrical stimulation is reversible. Additionally, stimulation parameters may be adjusted to minimize side effects while maintaining efficacy; such “fine tuning” is unavailable when producing a lesion. An implantable chronic stimulation device for deep brain stimulation (DBS) is available and similar systems are under development.

Other areas of the brain exhibit decreased neural activity in some patients with movement disorders. For instance, some Parkinson's disease patients demonstrate decreased neural activity in parts of the caudate and putamen (collectively named the striatum), external portions of the Globus Pallidus (GPe), and maybe also in portions of the thalamus.

A recent report presents early positive results for the application of extradural motor cortex stimulation for the treatment of movement disorders. [See Canavero, et al. “Extradural motor cortex stimulation for advanced Parkinson's disease: case report.” Movement Disorders 2000 January; 15(1): 169-171.] Low frequency stimulation at the extradural motor cortex was effective in relieving tremor, and offers several potential advantages over DBS. Unilateral motor cortex stimulation appears to treat tremor bilaterally, unlike DBS. Additionally, the efficacious stimulation settings reported were 25 Hz, 180 μsec, and 3 volts. This frequency is 5-10 times less than required for DBS, so will significantly increase the device lifetime and time between recharging.

However, the system used in the report (manufactured by Medtronic, Inc. of Minneapolis, Minn.) has several problems that make it an unacceptable option for some patients. It requires a significant surgical procedure for implantation, as the implantable pulse generator (IPG), a major component of the system containing the stimulation electronics and power source, must be implanted in the thorax and connected via a lead that runs in a subcutaneous tunnel through the chest, neck, and head to an electrode in an extradural location under the skull. Additionally, the IPG is bulky, which may produce an unsightly bulge at the implant site (e.g., the chest), especially for thin patients. Finally, the system is powered by a primary battery, which lasts only 3-4 years under normal operation. When the battery ceases to provide sufficient energy to adequately power the system, the patient must undergo an additional surgery in order to replace the IPG.

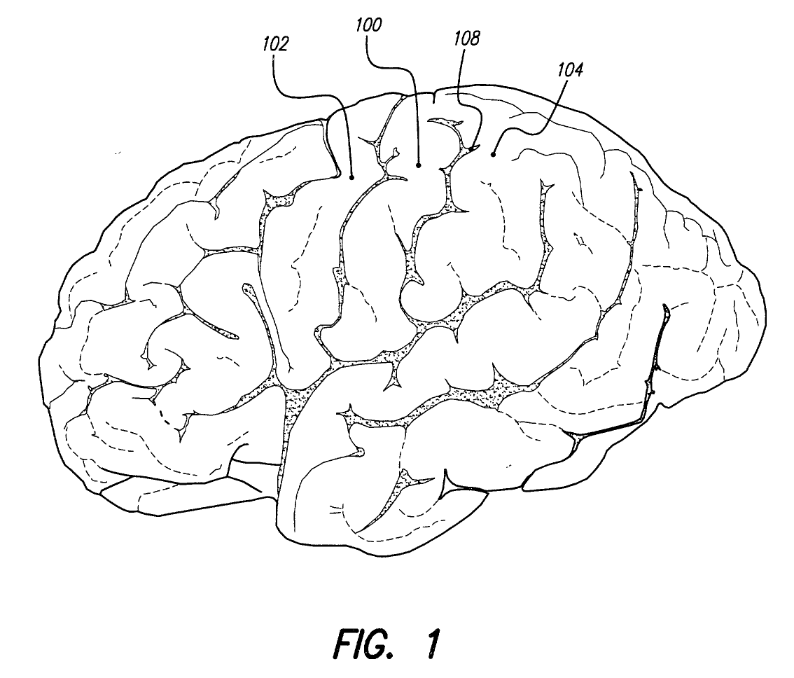

FIG. 1 shows the motor cortex 100 (which includes the precentral gyrus), premotor cortex 102, and the sensory cortex 104 (which includes the postcentral gyrus). As can be seen, motor cortex 100 lies on the outermost region of the brain, along the top and sides of the skull, and is the most posterior portion of the frontal lobe, lying just anterior to the central sulcus 108 (also known as the central fissure).

Motor cortex 100, which essentially consists of the precentral gyrus, transmits motor signals to all areas of the body. The signal origination points are somatotopically organized, so that stimulating one portion of the motor cortex produces a movement in the upper arm, while stimulating an adjacent portion may produce a movement in the lower arm. The premotor cortex 102 and the sensory cortex 104 are located adjacent the motor cortex 100, and are intimately connected to motor cortex functions. Stimulation may be applied to the these areas to suppress movements.

Thus, via mechanisms described in more detail herein, the present invention provides electrical stimulation and/or stimulating drugs to the motor cortex 100, the premotor cortex 102, and/or the sensory cortex 104 to adjust the level of neural activity in these portions of the cerebral cortex and/or in other areas of the brain affected by stimulation to these portions of the cortex, thereby treating or preventing motor disorders. For instance, for patients who demonstrate increased neural activity of the internal portion of the Globus Pallidus (GPi), the Subthalamic Nucleus (STN), and/or the striatum, inhibitory stimulation may be applied to the motor cortex 100, the premotor cortex 102, and/or the sensory cortex 104. On the other hand, for patients who exhibit decreased neural activity of the external portion of the Globus Pallidus (GPe), striatum, and/or portions of the thalamus, excitatory stimulation may be applied to the motor cortex 100, the premotor cortex 102, and/or the sensory cortex 104.

Herein, stimulating drugs comprise medications, anesthetic agents, synthetic or natural hormones, neurotransmitters, interleukins, cytokines, lymphokines, chemokines, growth factors, and other intracellular and intercellular chemical signals and messengers, and the like. Certain neurotransmitters, hormones, and other drugs are excitatory for some tissues, yet are inhibitory to other tissues. Therefore, where, herein, a drug is referred to as an “excitatory” drug, this means that the drug is acting in an excitatory manner, although it may act in an inhibitory manner in other circumstances and/or locations. Similarly, where an “inhibitory” drug is mentioned, this drug is acting in an inhibitory manner, although in other circumstances and/or locations, it may be an “excitatory” drug. In addition, stimulation of an area herein may include stimulation of cell bodies and axons in the area.

In some alternatives, stimulation is provided by at least one system control unit (SCU) that is an implantable signal generator connected to an electrode(s) and/or an implantable pump connected to a catheter(s). These systems deliver electrical stimulation and/or one or more stimulating drugs to the motor cortex. One or more electrodes are surgically implanted to provide electrical stimulation, and/or one or more catheters are surgically implanted to infuse the stimulating drug(s).

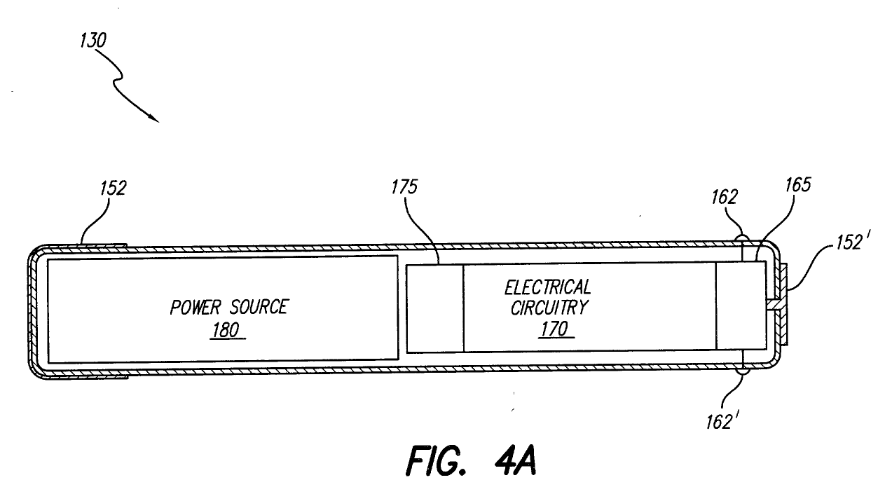

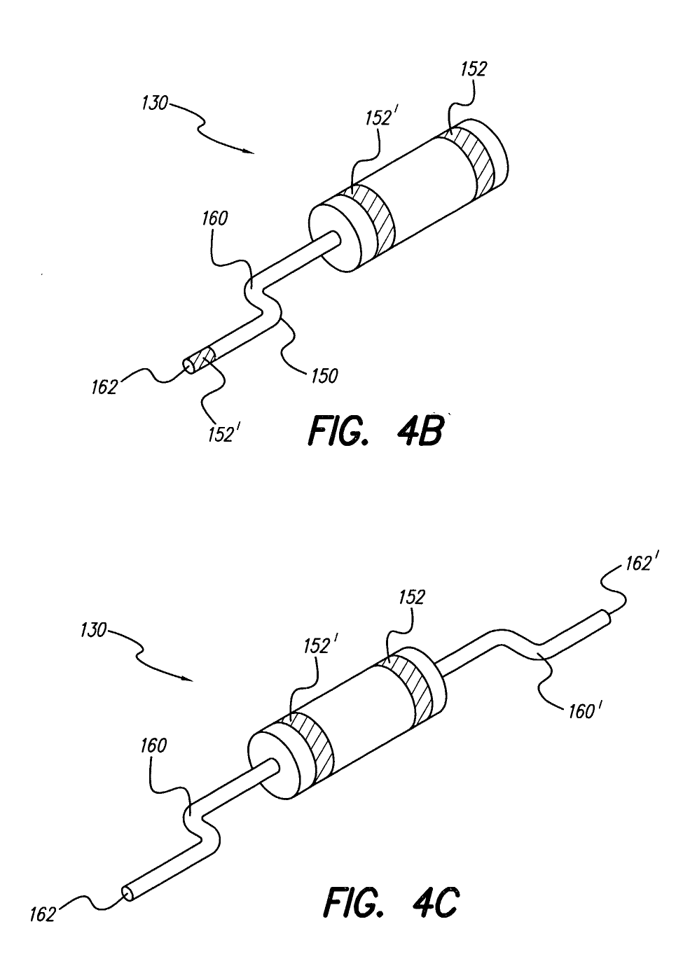

In various alternatives, stimulation is provided by one or more SCUs that are small, implantable stimulators, referred to herein as microstimulators. The microstimulators of the present invention may be similar to or of the type referred to as BION® devices (see FIGS. 4A, 4B, and 4C). The following documents describe various details associated with the manufacture, operation and use of BION implantable microstimulators, and are all incorporated herein by reference:

| Application/ | Filing/ | |
|---|---|---|
| Patent/ | Publication | |
| Publication No. | Date | Title |
| U.S. Pat. No. | Issued | Implantable Microstimulator |
| 5,193,539 | Mar 16, 1993 | |
| U.S. Pat. No. | Issued | Structure and Method of |
| 5,193,540 | Mar 16, 1993 | Manufacture of an Implantable |
| | | Microstimulator |
| U.S. Pat. No. | Issued | Implantable Device Having an |
| 5,312,439 | May 17, 1994 | Electrolytic Storage Electrode |
| U.S. Pat. No. | Issued | Implantable Microstimulator |
| 5,324,316 | Jun. 28, 1994 | |
| U.S. Pat. No. | Issued | Structure and Method of |
| 5,405,367 | Apr. 11, 1995 | Manufacture of an Implantable |
| | | Microstimulator |
| PCT Publication | Published | Battery-Powered Patient |
| WO 98/37926 | Sep. 3, 1998 | Implantable Device |
| PCT Publication | Published | System of Implantable Devices |
| WO 98/43700 | Oct. 8, 1998 | For Monitoring and/or Affecting |
| | | Body Parameters |
| PCT Publication | Published | System of Implantable Devices |
| WO 98/43701 | Oct. 8, 1998 | For Monitoring and/or Affecting |
| | | Body Parameters |
| U.S. Pat. No. | Issued | Improved Implantable Microstimu- |
| 6,051,017 | Apr. 18, 2000 | lator and Systems Employing Same |
| | Published | Micromodular Implants to Provide |
| | September, | Electrical Stimulation of Para- |
| | 1997 | lyzed Muscles and Limbs, by |
| | | Cameron, et al., published in |
| | | IEEE Transactions on Biomedical |
| | | Engineering, Vol. 44, No. 9, |
| | | pages 781-790. |

As shown in FIGS. 4A, 4B, and 4C, microstimulator SCUs 130 may include a narrow, elongated capsule containing electronic circuitry 170 connected to electrodes 152 and 152′, which may pass through the walls of the capsule at either end. Alternatively, electrodes 152 and/or 152′ may be built into the case and/or arranged on a catheter 160 or at the end of a lead, as shown in FIG. 4B. As detailed in the referenced patent publications, electrodes 152 and 152′ generally comprise a stimulating electrode (to be placed close to the target tissue) and an indifferent electrode (for completing the circuit). Other configurations of microstimulator SCU 130 are possible, as is evident from the above-referenced publications, and as described in more detail herein.

Certain configurations of implantable microstimulator SCU 130 are sufficiently small to permit placement in or adjacent to the structures to be stimulated. For instance, in these configurations, the microstimulator capsule may have a diameter of about 4-5 mm, or only about 3 mm, or even less than about 3 mm. In these configurations, capsule length may be about 25-35 mm, or only about 20-25 mm, or even less than about 20 mm. The shape of the microstimulator may be determined by the structure of the desired target, the surrounding area, and the method of implantation. A thin, elongated cylinder with electrodes at the ends, as shown in FIGS. 4A, 4B, and 4C, is one possible configuration, but other shapes, such as cylinders, disks, spheres, and helical structures, are possible, as are additional electrodes, infusion outlets, leads, and/or catheters.

Microstimulator SCU 130, when certain configurations are used, may be implanted with a surgical tool such as a tool specially designed for the purpose, or with a hypodermic needle, or the like. Alternatively, microstimulator SCU 130 may be implanted via conventional surgical methods (e.g., via a small incision), or may be placed using endoscopic or laparoscopic techniques. A more complicated surgical procedure may be required, e.g., for fixing the microstimulator in place.

The external surfaces of microstimulator SCU 130 may advantageously be composed of biocompatible materials. The SCU capsule may be made of, for instance, glass, ceramic, or other material that provides a hermetic package that will exclude water vapor but permit passage of electromagnetic fields used to transmit data and/or power. Electrodes 152 and 152′ may be made of a noble or refractory metal or compound, such as platinum, iridium, tantalum, titanium, titanium nitride, niobium or alloys of any of these, in order to avoid corrosion or electrolysis which could damage the surrounding tissues and the device.

In certain embodiments of the instant invention, microstimulator SCU 130 comprises two leadless electrodes. However, either or both electrodes 152 and 152′ may alternatively be located at the ends of short, flexible leads as described in U.S. patent application Ser. No. 09/624,130, filed Jul. 24, 2000, which is incorporated herein by reference in its entirety. The use of such leads permits, among other things, electrical stimulation to be directed more locally to targeted tissue(s) a short distance from the surgical fixation of the bulk of microstimulator SCU 130, while allowing most elements of the microstimulator to be located in a more surgically convenient site. This minimizes the distance traversed and the surgical planes crossed by the device and any lead(s). In most uses of this invention, the leads are no longer than about 150 mm.

As mentioned earlier, stimulation is provided in accordance with the teachings of the present invention by electrical stimulation and/or one or more stimulating drugs delivered to the body by one or more system control units (SCUs). In the case of electrical stimulation only, SCUs include a microstimulator and/or an implantable pulse/signal generator (IPG), or the like. In the case of drug infusion only, an SCU comprises an implantable pump or the like. In cases requiring both electrical stimulation and drug infusion, more than one SCU may be used. Alternatively, when needed and/or desired, an SCU provides both electrical stimulation and one or more stimulating drugs.

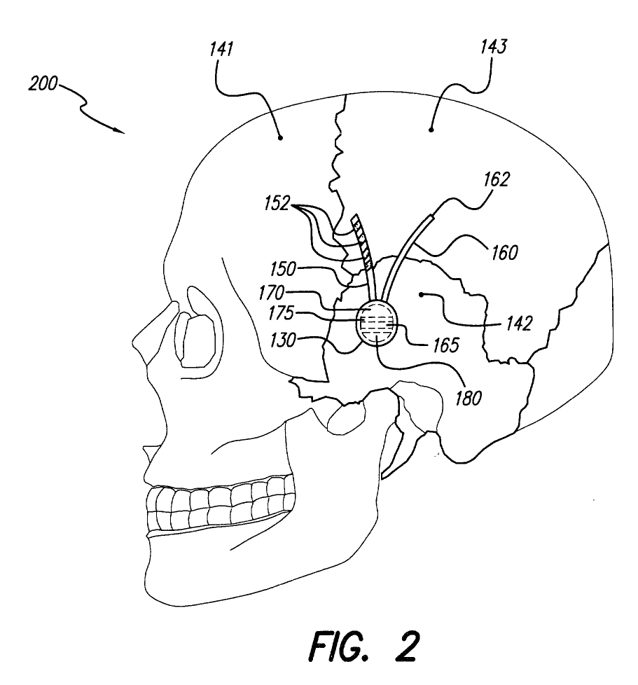

As seen in FIG. 2, some embodiments of SCU 130 may be (but are not necessarily) implanted beneath the scalp, such as in a surgically-created shallow depression or opening in the skull of patient 200, for instance, in parietal bone 141, temporal bone 142, or frontal bone 143. In several embodiments, SCU 130 conforms to the profile of surrounding tissue(s) and/or bone(s), and is small and compact. This may minimize pressure applied to the skin or scalp, which pressure may result in skin erosion or infection. In various embodiments, SCU 160 has a diameter of about 75 mm, or only about 65 mm, or even less than about 55 mm. In such configurations, SCU thickness (e.g., depth into the skull) may be about 10-12 mm, or even less than about 10 mm.

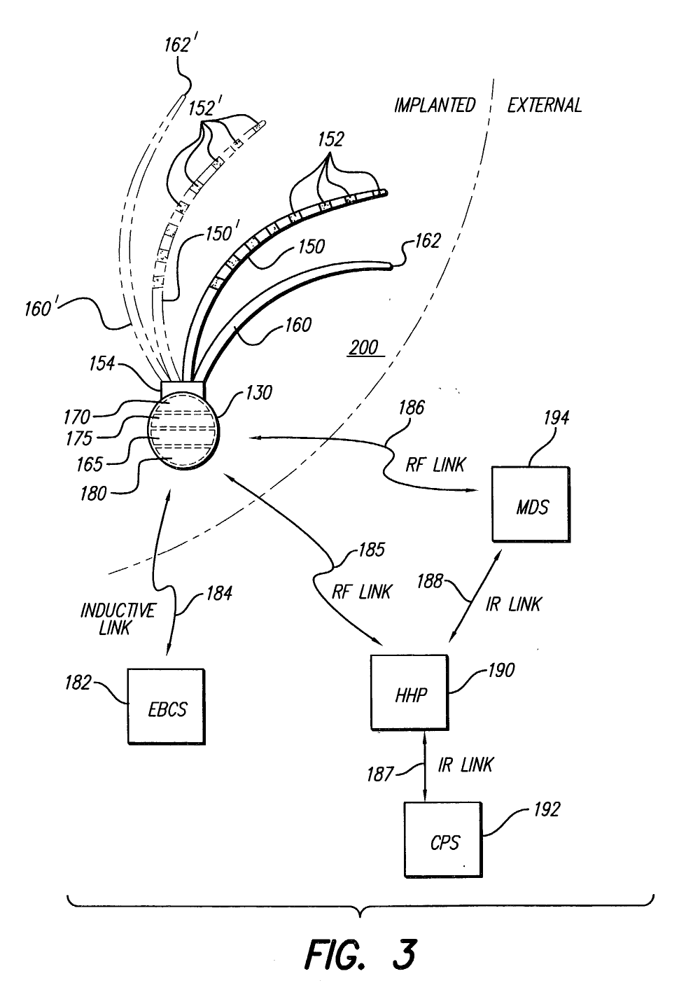

As seen in the embodiments depicted in FIG. 3, one or more electrode leads 150 and/or catheters 160 attached to SCU 130 run subcutaneously, for instance, in a surgically-created shallow groove(s) in the skull, to an opening(s) in the skull, and pass through the opening(s) into the extradural area under the skull. Recessed placement of the SCU and the lead(s) and/or catheter(s) may decrease the likelihood of erosion of the overlying skin, and may minimize any cosmetic impact.

In embodiments such as in FIG. 3, electrode(s) 152 are carried on lead 150 having a proximal end coupled to SCU 130. The lead contains wires electrically connecting electrodes 152 to SCU 130. SCU 130 contains electrical components 170 that produce electrical stimulation pulses that travel through the wires of lead 150 and are delivered to electrodes 152, and thus to the tissue surrounding electrodes 152. To protect the electrical components inside SCU 130, some or all of the case of the SCU may be hermetically sealed. For additional protection against, e.g., impact, the case may be made of metal (e.g. titanium) or ceramic, which materials are also, advantageously, biocompatible. In addition, SCU 130 may be configured to be Magnetic Resonance Imaging (MRI) compatible.

In some alternatives, the electrical stimulation may be provided as described in International Patent Application Serial Number PCT/US01/04417 (the '417 application), filed Feb. 12, 2001, and published Aug. 23, 2001 as WO 01/60450, which application is incorporated herein by reference in its entirety. As such, the electrical stimulation of the present invention may be as provided in this PCT application, which is directed to a “Deep Brain Stimulation System for the Treatment of Parkinson's Disease or Other Disorders”.

In the case of treatment alternatively or additionally constituting drug infusion, SCU 130 (which herein refers to IPGs, implantable pumps, IPG/pump combinations, microstimulators for drug and/or electrical stimulation, and/or other alternative devices described herein) may contain at least one pump 165 for storing and dispensing one or more drugs through outlet(s) 162/162′ and/or catheter(s) 160/160′. When a catheter is used, it includes at least one infusion outlet 162, usually positioned at least at a distal end, while a proximal end of the catheter is connected to SCU 130.

According to some embodiments of the invention, such as described in the previously referenced '417 application and as depicted in FIG. 3, at least one lead 150 is attached to SCU 130, via a suitable connector 154, if necessary. Each lead includes at least two electrodes 152, and may include as many as sixteen or more electrodes 152. Additional leads 150′ and/or catheter(s) 160′ may be attached to SCU 130. Hence, FIG. 3 shows (in phantom lines) a second catheter 160′, and a second lead 150′, having electrodes 152′ thereon, also attached to SCU 130. Similarly, the SCU 130 of FIGS. 4A, 4B, and 4C have outlets 162, 162′ for infusing a stimulating drug(s) and electrodes 152, 152′ for applying electrical stimulation.

Cylindrical lead(s) 150 of certain embodiments of the present invention may be less than 5 mm in diameter, or even less than about 1.5 mm in diameter. In embodiments using one or more paddle-shaped leads, lead(s) 150 may be less than 15 mm in width, and less than 1.5 mm in thickness. Electrodes 152, 152′ on leads 150, 150′ may be arranged as an array, for instance, as two or more collinear electrodes, or even as four or more collinear electrodes, or they may not be collinear. A tip electrode may also be supplied at the distal end of one or more leads.

In some embodiments, SCU 130 is programmable to produce either monopolar electrical stimulation, e.g., using the SCU case as an indifferent electrode, or bipolar electrical stimulation, e.g., using one of the electrodes of the electrode array as an indifferent electrode. Some embodiments of SCU 130 have at least four channels and drive up to sixteen electrodes or more.

SCU 130 contains, when necessary and/or desired, electronic circuitry 170 for receiving data and/or power from outside the body by inductive, radio frequency (RF), or other electromagnetic coupling. In some embodiments, electronic circuitry 170 includes an inductive coil for receiving and transmitting RF data and/or power, an integrated circuit (IC) chip for decoding and storing stimulation parameters and generating stimulation pulses (either intermittent or continuous), and additional discrete electronic components required to complete the electronic circuit functions, e.g. capacitor(s), resistor(s), coil(s), and the like.

SCU 130 also includes, when necessary and/or desired, a programmable memory 175 for storing a set(s) of data, stimulation, and control parameters. Among other things, memory 164 may allow electrical and/or drug stimulation to be adjusted to settings that are safe and efficacious with minimal discomfort for each individual. Specific parameters may provide therapeutic advantages for various types and severities of movement disorders. For instance, some patients may respond favorably to intermittent stimulation, while others may require continuous treatment for relief. In some embodiments, electrical and drug stimulation parameters are controlled independently, e.g., continuous electrical stimulation and no drug stimulation. However, in some instances, they may advantageously be coupled, e.g., electrical stimulation may be programmed to occur only during drug infusion.

In addition, different stimulation parameters may have different effects on neural tissue. Therefore, parameters may be chosen to target specific neural populations and/or to exclude others, or to increase neural activity in specific neural populations and/or to decrease neural activity in others. For example, relatively low frequency neurostimulation (i.e., less than about 100-150 Hz) typically has an excitatory effect on surrounding neural tissue, leading to increased neural activity, whereas relatively high frequency neurostimulation (i.e., greater than about 100-150 Hz) may have an inhibitory effect, leading to decreased neural activity. Similarly, excitatory neurotransmitters (e.g., glutamate, glutamate receptor agonist(s), dopamine, norepinephrine, epinephrine, acetylcholine, serotonin), agonists thereof, and agents that act to increase levels of an excitatory neurotransmitter(s) (e.g., edrophonium, Mestinon) generally have an excitatory effect on neural tissue, while inhibitory neurotransmitters (e.g., dopamine, glycine, and gamma-aminobutyric acid, a.k.a. GABA), agonists thereof (e.g., benzodiazepines, such as diazepam, or barbiturates), and agents that act to increase levels of an inhibitory neurotransmitter(s) generally have an inhibitory effect. (Dopamine acts as an excitatory neurotransmitter in some locations and circumstances, and as an inhibitory neurotransmitter in other locations and circumstances.) However, antagonists of inhibitory neurotransmitters (e.g., bicuculline) and agents that act to decrease levels of an inhibitory neurotransmitter(s) have been demonstrated to excite neural tissue, leading to increased neural activity. Similarly, excitatory neurotransmitter antagonists (e.g. prazosin, metoprolol) and agents that decrease levels of excitatory neurotransmitter(s) (e.g., acetylcholinesterase) may inhibit neural activity.

Some embodiments of SCU 130 also include a power source and/or power storage device 180. Possible power options for a stimulation device of the present invention, described in more detail below, include but are not limited to an external power source coupled to the stimulation device, e.g., via an RF link, a self-contained power source utilizing any suitable means of generation or storage of energy (e.g., a primary battery, a replenishable or rechargeable battery such as a lithium ion battery, an electrolytic capacitor, a super- or ultra-capacitor, or the like), and if the self-contained power source is replenishable or rechargeable, means of replenishing or recharging the power source (e.g., an RF link, an optical link, a thermal link, or other energy-coupling link).

In embodiments such as shown in FIG. 3, SCU 130 includes a rechargeable battery as a power source/storage device 180. The battery is recharged, as required, from an external battery charging system (EBCS) 182, typically through an inductive link 184. In these embodiments, and as explained more fully in the earlier referenced '417 PCT application, SCU 130 includes a processor and other electronic circuitry 170 that allow it to generate stimulation pulses that are applied to the patient 200 through electrodes 152 and/or outlet(s) 162 in accordance with a program and stimulation parameters stored in programmable memory 175. Stimulation pulses of drugs include various types and/or rates of infusion, such as intermittent infusion, infusion at a constant rate, and bolus infusion.

According to certain embodiments of the invention, an SCU operates independently. According to various embodiments of the invention, an SCU operates in a coordinated manner with other SCU(s), other implanted device(s), and/or other device(s) external to the patient's body. For instance, an SCU may control or operate under the control of another implanted SCU(s), other implanted device(s), and/or other device(s) external to the patient's body. An SCU may communicate with other implanted SCUs, other implanted devices, and/or devices external to a patient's body via, e.g., an RF link, an ultrasonic link, a thermal link, and/or an optical link. Specifically, an SCU may communicate with an external remote control (e.g., patient and/or physician programmer) that is capable of sending commands and/or data to an SCU and that may also be capable of receiving commands and/or data from an SCU.

For example, some embodiments of SCU 130 of the present invention may be activated and deactivated, programmed and tested through a hand held programmer (HHP) 190 (which may also be referred to as a patient programmer and may be, but is not necessarily, hand held), a clinician programming system (CPS) 192 (which may also be hand held), and/or a manufacturing and diagnostic system (MDS) 194 (which may also be hand held). HHP 190 may be coupled to SCU 130 via an RF link 185. Similarly, MDS 194 may be coupled to SCU 130 via another RF link 186. In a like manner, CPS 192 may be coupled to HHP 190 via an, infra-red link 187; and MDS 194 may be coupled to HHP 190 via another infra-red link 188. Other types of telecommunicative links, other than RF or infra-red may also be used for this purpose. Through these links, CPS 192, for example, may be coupled through HHP 190 to SCU 130 for programming or diagnostic purposes. MDS 194 may also be coupled to SCU 130, either directly through RF link 186, or indirectly through the IR link 188, HHP 190, and RF link 185.

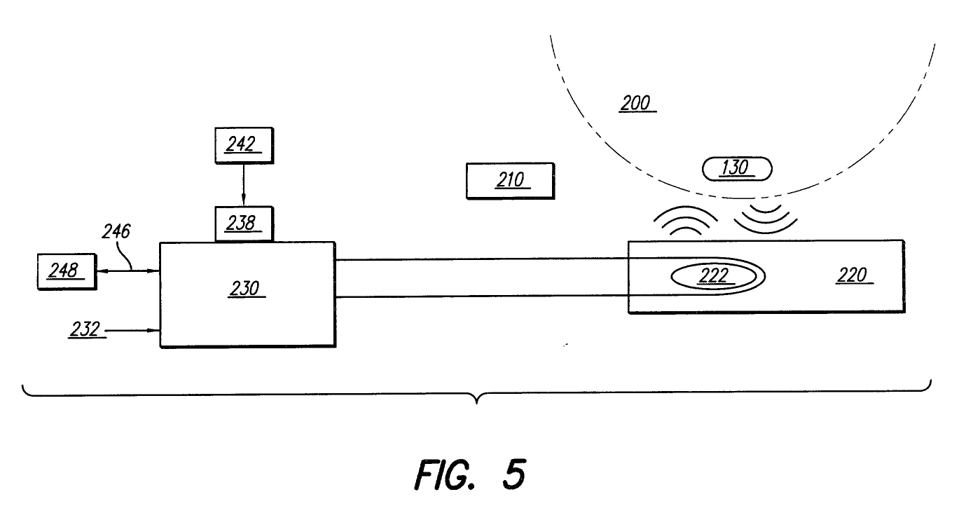

In certain embodiments, using for example, a BION microstimulator(s) as described in the above referenced patents, and as illustrated in FIG. 5, the patient 200 switches SCU 130 on and off by use of controller 210, which may be handheld. SCU 130 is operated by controller 210 by any of various means, including sensing the proximity of a permanent magnet located in controller 210, sensing RF transmissions from controller 210, or the like.

External components for programming and/or providing power to various embodiments of SCU 130 are also illustrated in FIG. 5. When communication with such an SCU 130 is desired, patient 200 is positioned on or near external appliance 220, which appliance contains one or more inductive coils 222 or other means of communication (e.g., RF transmitter and receiver). External appliance 220 is connected to or is a part of external electronic circuitry appliance 230 which may receive power 232 from a conventional power source. External appliance 230 contains manual input means 238, e.g., a keypad, whereby the patient 200 or a caregiver 242 may request changes in electrical and/or drug stimulation parameters produced during the normal operation of SCU 130. In these embodiments, manual input means 238 includes various electro-mechanical switches and/or visual display devices that provide the patient and/or caregiver with information about the status and prior programming of SCU 130.

Alternatively or additionally, external electronic appliance 230 is provided with an electronic interface means 246 for interacting with other computing means 248, such as by a serial interface cable or infrared link to a personal computer or to a telephone modem or the like. Such interface means 246 may permit a clinician to monitor the status of the implant and prescribe new stimulation parameters from a remote location.

The external appliance(s) may be embedded in a cushion, pillow, or hat. Other possibilities exist, including a head band, patch, or other structure(s) that may be affixed to the patient's body or clothing. External appliances may include a package that can be, e.g., worn on the belt, may include an extension to a transmission coil affixed, e.g., with a velcro band or adhesive, or may be combinations of these or other structures able to perform the functions described herein.

In order to help determine the strength and/or duration of electrical stimulation and/or the amount and/or type(s) of stimulating drug(s) required to produce the desired effect, in some embodiments, a patient's response to and/or need for treatment is sensed. For example, when electrodes and/or infusion outlet(s) of SCU 130 are implanted on or near the motor cortex 100, signals from an EEG built into SCU 130 may be recorded. (As used herein, “near” and “adjacent” mean as close as reasonably possible to targeted tissue, including touching or even being positioned within the tissue, but in general, may be as far as about 150 mm from the target tissue.)

Alternatively, an “SCU” dedicated to sensory processes communicates with an SCU providing stimulation pulses. The implant circuitry 170 may, if necessary, amplify and transmit these sensed signals, which may be digital or analog. Besides measuring the electrical activity of a neural population (e.g., EEG), other methods of determining the required electrical and/or drug stimulation include measuring neurotransmitter levels and/or their associated breakdown product levels, hormone levels, or other substances, such as dopamine levels, interleukins, cytokines, lymphokines, chemokines, growth factors, enzymes, medication and/or other drug levels, and/or levels of any other bloodborne substance(s), and/or changes in one or more of these may be sensed, using, e.g., one or more Chemically Sensitive Field-Effect Transistors (CHEMFETs) such as Enzyme-Selective Field-Effect Transistors (ENFETs) or Ion-Sensitive Field-Effect Transistors (ISFETs, as are available from Sentron CMT of Enschede, The Netherlands). Other methods are mentioned herein, and yet others will be evident to those of skill in the field upon review of the present disclosure. The sensing may occur during stimulation or during a temporary suspension of stimulation. The sensed information may be used to control stimulation parameters in a closed-loop manner.

For instance, in several embodiments of the present invention, a first and second “SCU” are provided. The second “SCU” periodically (e.g. once per minute) records limb electromyograph (EMG) activity, which it transmits to the first SCU. The first SCU uses the sensed information to adjust electrical and/or drug stimulation parameters according to an algorithm programmed, e.g., by a physician. For example, the amplitude and/or frequency of electrical stimulation may be increased in response to increased rhythmic EMG activity. In some alternatives, one SCU performs both the sensing and stimulating functions, as discussed in more detail presently.

While an SCU 130 may also incorporate means of sensing symptoms or other prognostic or diagnostic indicators of movement disorders, e.g., via EMG, it may alternatively or additionally be desirable to use a separate or specialized implantable device to record and telemeter physiological conditions/responses in order to adjust electrical stimulation and/or drug infusion parameters. This information may be transmitted to an external device, such as external appliance 220, or may be transmitted directly to implanted SCU(s) 130. However, in some cases, it may not be necessary or desired to include a sensing function or device, in which case stimulation parameters are determined and refined, for instance, by patient feedback, or the like.

Thus, it is seen that in accordance with the present invention, one or more external appliances may be provided to interact with SCU 130, and may be used to accomplish, potentially among other things, one or more of the following functions:
Function 1: If necessary, transmit electrical power from the external electronic appliance 230 via appliance 220 to SCU 130 in order to power the device and/or recharge the power source/storage device 180. External electronic appliance 230 may include an automatic algorithm that adjusts electrical and/or drug stimulation parameters automatically whenever the SCU(s) 130 is/are recharged.
Function 2: Transmit data from the external appliance 230 via the external appliance 220 to SCU 130 in order to change the parameters of electrical and/or drug stimulation used by SCU 130.
Function 3: Transmit sensed data indicating a need for treatment or in response to stimulation from SCU 130 (e.g., EEG, neurotransmitter levels, or other activity) to external appliance 230 via external appliance 220.
Function 4: Transmit data indicating state of the SCU 130 (e.g., battery level, drug level, stimulation parameters, etc.) to external appliance 230 via external appliance 220.

By way of example, a treatment modality for movement disorders, e.g., Parkinson's disease, may be carried out according to the following sequence of procedures:
1. A first SCU 130 is implanted so that its electrodes 152 and/or infusion outlet 162 are located in or on or near the extradural motor cortex 100. If necessary or desired, electrodes 152′ and/or infusion outlets 162′ may additionally or alternatively be located extradurally or subdurally in/on or near the motor cortex 100, premotor cortex 102, or sensory cortex 104.
2. Using Function 2 described above (i.e., transmitting data) of external electronic appliance 230 and external appliance 220, first SCU 130 is commanded to produce a series of excitatory electrical stimulation pulses, possibly with gradually increasing amplitude, and possibly while infusing gradually increasing amounts of an excitatory cortical neurotransmitter agonist, e.g., glutamate.
3. After each stimulation pulse, series of pulses, or at some other predefined interval, any change in, e.g., electrical activity of a muscular population (e.g., rhythmic EMG) resulting from the electrical and/or drug stimulation is sensed, for instance, by one or more electrodes 152, 152′ or sensors of a second SCU 130, such as a microstimulator SCU 130, implanted in or on or near a muscle(s) of a limb, e.g., forearm extensor muscles. These responses are converted to data and telemetered out to external electronic appliance 230 via Function 3.
4. From the response data received at external appliance 230 from second SCU 130, or from some other assessment, the stimulus threshold for obtaining a response is determined and is used by a clinician 242 acting directly 238 or by other computing means 248 to transmit the desired electrical and/or drug stimulation parameters to first SCU 130 in accordance with Function 2.
5. When patient 200 desires to invoke electrical stimulation and/or drug infusion, patient 200 employs controller 210 to set first SCU 130 in a state where it delivers a prescribed stimulation pattern from a predetermined range of allowable stimulation patterns.
6. To cease electrical and/or drug stimulation, patient 200 employs controller 210 to turn off first SCU 130 and possibly also second SCU 130.
7. Periodically, the patient or caregiver recharges the power source/storage device 180 of first and/or second SCU 130, if necessary, in accordance with Function 1 described above (i.e., transmit electrical power).

In another example, a treatment for movement disorders, e.g., Parkinson's disease, may be carried out according to the following sequence of procedures:
1. An SCU 130 is implanted so that its electrodes 152 and possibly also infusion outlet 162 are located in or on or near the extradural motor cortex 100 (e.g., a Bion may be located subcutaneously above the skull).
2. Using Function 2 described above (i.e., transmitting data) of external electronic appliance 230 and external appliance 220, SCU 130 is commanded to produce a series of excitatory electrical stimulation pulses, possibly with gradually increasing amplitude, and possibly while infusing gradually increasing amounts of an excitatory cortical neurotransmitter agonist, e.g., glutamate.
3. After each stimulation pulse, series of pulses, or at some other predefined interval, any change in electrical activity of a neural population (e.g., rhythmic EEG) of the motor cortex 100 resulting from the electrical and/or drug stimulation is sensed, for instance, by one or more of the electrodes 152 of SCU 130. These responses are converted to data and telemetered out to external electronic appliance 230 via Function 3.
4. From the response data received at external appliance 230 from SCU 130, or from some other assessment, the stimulus threshold for obtaining a response is determined and is used by a clinician 242 acting directly 238 or by other computing means 248 to transmit the desired electrical and/or drug stimulation parameters to SCU 130 in accordance with Function 2.
5. When patient 200 desires to invoke electrical stimulation and/or drug infusion, patient 200 employs controller 210 to set SCU 130 in a state where it delivers a prescribed stimulation pattern from a predetermined range of allowable stimulation patterns.
6. To cease electrical and/or drug stimulation, patient 200 employs controller 210 to turn off SCU 130.
7. Periodically, the patient or caregiver recharges the power source/storage device 180 of SCU 130, if necessary, in accordance with Function 1 described above (i.e., transmit electrical power).

For the treatment of any of the various types and severities of movement disorders, it may be desirable to modify or adjust the algorithmic functions performed by the implanted and/or external components, as well as the surgical approaches, in ways that would be obvious to skilled practitioners of these arts. For example, in some situations, it may be desirable to employ more than one SCU 130, each of which could be separately controlled by means of a digital address. Multiple channels and/or multiple patterns of electrical and/or drug stimulation might thereby be programmed by the clinician and controlled by the patient in order to, for instance, deal with complex or multiple symptoms or conditions, such as Parkinson's disease coupled with side effects from medication, e.g., dyskinesia.

In some embodiments discussed earlier, SCU 130, or a group of two or more SCUs, is controlled via closed-loop operation. A need for and/or response to stimulation is sensed via SCU 130, or by an additional SCU (which may or may not be dedicated to the sensing function), or by another implanted or external device. If necessary, the sensed information is transmitted to SCU 130. In some cases, the sensing and stimulating are performed by one SCU. In some embodiments, the parameters used by SCU 130 are automatically adjusted based on the sensed information. Thus, the electrical and/or drug stimulation parameters are adjusted in a closed-loop manner to provide stimulation tailored to the need for and/or response to the electrical and/or drug stimulation.

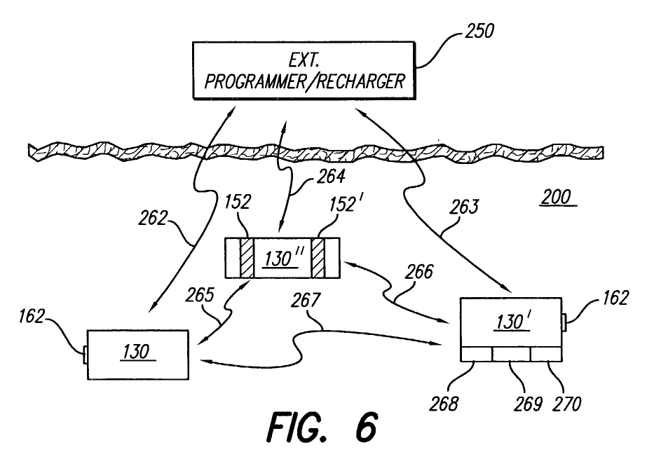

For instance, as shown in the examples of FIG. 6, a first SCU 130, implanted beneath the skin of the patient 200, provides a first medication or substance; a second SCU 130′ provides a second medication or substance; and a third SCU 130″ provides electrical stimulation via electrodes 152 and 152′. As mentioned earlier, the implanted devices may operate independently or may operate in a coordinated manner with other similar implanted devices, other implanted devices, or other devices external to the patient's body, as shown by the control lines 262, 263 and 264 in FIG. 6. That is, in accordance with certain embodiments of the invention, the external controller 250 controls the operation of each of the implanted devices 130, 130′ and 130″. According to various embodiments of the invention, an implanted device, e.g. SCU 130, may control or operate under the control of another implanted device(s), e.g. SCU 130′ and/or SCU 130″. That is, a device made in accordance with the invention may communicate with other implanted stimulators, other implanted devices, and/or devices external to a patient's body, e.g., via an RF link, an ultrasonic link, a thermal link, an optical link, or the like. Specifically, as illustrated in FIG. 6, SCU 130, 130′, and/or 130″, made in accordance with the invention, may communicate with an external remote control (e.g., patient and/or physician programmer 250) that is capable of sending commands and/or data to implanted devices and that may also be capable of receiving commands and/or data from implanted devices.

A drug infusion stimulator made in accordance with the invention may incorporate communication means for communicating with one or more external or site-specific drug delivery devices, and, further, may have the control flexibility to synchronize and control the duration of drug delivery. The associated drug delivery device typically provides a feedback signal that lets the control device know it has received and understood commands. The communication signal between the implanted stimulator and the drug delivery device may be encoded to prevent the accidental or inadvertent delivery of drugs by other signals.

An SCU made in accordance with the invention thus incorporates, in some embodiments, first sensing means 268 for sensing therapeutic effects, clinical variables, or other indicators of the state of the patient, such as EMG, EEG, or the like. The stimulator additionally or alternatively incorporates second means 269 for sensing neurotransmitter levels and/or their associated breakdown product levels, medication levels and/or other drug levels, hormone, enzyme, interleukin, cytokine, lymphokine, chemokine, and/or growth factor levels and/or changes in these or other substances in the blood plasma, local interstitial fluid, and/or cerebrospinal fluid. The stimulator additionally or alternatively incorporates third means 270 for sensing electrical current levels and/or waveforms supplied by another source of electrical energy. Sensed information may be used to control infusion and/or electrical parameters in a closed loop manner, as shown by control lines 266, 267, and 265. Thus, the sensing means may be incorporated into a device that also includes electrical and/or drug stimulation, or the sensing means (that may or may not have stimulating means), may communicate the sensed information to another device(s) with stimulating means.

According to some embodiments of the invention, the electrical and/or drug stimulation decreases activity of one or more of those areas of the brain that exhibit chronic increased activity, relative to control subjects, in patients experiencing a movement disorder(s). These areas may include one or more of the motor cortex 100, premotor cortex 102, sensory cortex 104, internal portion of the Globus Pallidus (GPi), Subthalamic Nucleus (STN), and/or striatum. Such inhibitory stimulation is likely to be produced by relatively high-frequency electrical stimulation (e.g., greater than about 100-150 Hz), an excitatory neurotransmitter antagonist(s) (e.g. prazosin, metoprolol), an inhibitory neurotransmitter(s) (e.g., GABA), an agonist thereof, an agent that increases the level of an inhibitory neurotransmitter, an agent that decreases the level of an excitatory neurotransmitter, a local anesthetic agent (e.g., lidocaine), and/or an analgesic medication. This stimulation may be applied through the skull, extradurally, or subdurally to one or more of the motor cortex 100, premotor cortex 102, or sensory cortex 104 to treat movement disorder(s).

According to other embodiments of the invention, the electrical and/or drug stimulation increases activity of one or more of those areas of the brain that exhibit chronic decreased activity, relative to control subjects, in patients experiencing a movement disorder(s), thereby treating or preventing such disorder(s) and/or the symptoms and/or pathological consequences thereof. These areas may include one or more of the motor cortex 100, premotor cortex 102, sensory cortex 104, external portion of the Globus Pallidus (GPe), portions of the thalamus, and/or striatum. Such excitatory stimulation is likely to be produced by low-frequency electrical stimulation (e.g., less than about 100-150 Hz), an excitatory neurotransmitter (e.g., glutamate), an excitatory cortical neurotransmitter agonist (e.g., glutamate receptor agonist, bethanechol, norepinephrine), an inhibitory neurotransmitter antagonist(s) (e.g., bicuculline), an agent that increases the level of an excitatory neurotransmitter (e.g., edrophonium), and/or an agent that decreases the level of an inhibitory neurotransmitter. This stimulation may be applied through the skull, extradurally, or subdurally to one or more of the motor cortex 100, premotor cortex 102, or sensory cortex 104 to treat movement disorder(s).

In various embodiments, sensing means described earlier may be used to orchestrate first the activation of SCU(s) targeting an area(s) of the extradural motor cortex, and then, when appropriate, SCU(s) targeting another area(s) and/or by different means. Alternatively, this orchestration may be programmed, and not based on a sensed condition.

While the invention herein disclosed has been described by means of specific embodiments and applications thereof, numerous modifications and variations could be made thereto by those skilled in the art without departing from the scope of the invention set forth in the claims.

1-2. (canceled)

3. A method of treating a Patient with a movement disorder, comprising:
implanting in the patient's head at least one leadless system control unit for controlling delivery of at least one stimulus comprising an electrical stimulation current and one or more drugs, said system control unit being connected to at least one infusion outlet and having an outer casing and a number of electrodes disposed on the casing;
applying the electrical stimulation current via said electrodes to at least one area of the brain affecting a movement disorder in order to at least in part alleviate the movement disorder of the patient being treated;
delivering the one or more drugs through the at least one outlet to the at least one area of the brain affecting the movement disorder; and
selecting the at least one area of the brain from at least one of a motor cortex, a premotor cortex, and a sensory vortex.

4. (canceled)

5. The method of claim 3 wherein the at least one stimulus decreases activity of at least one area of the brain affecting pain that exhibits chronic increased activity.

6. The method of claim 3 wherein the electrical stimulation current is delivered at a frequency greater than or equal to about 100 Hz.

7. The method of claim 3 wherein the one or more drugs comprise at least one or more of an excitatory neurotransmitter antagonist, an inhibitory neurotransmitter, an inhibitory neurotrarismitter agonist, an agent that increases a level of an inhibitory neurotransmitter, an agent that decreases a level of an excitatory neurotransmitter, a local anesthetic agent, and an analgesic medication.

8. The method of claim 3 wherein the one or more drugs comprise at least one or more of dopamine, GABA, and a GABA agonist.

9. The method of claim 3 wherein the at least one stimulus increases activity of the at least one area of the brain that exhibits chronic decreased activity.

10. The method of claim 3 wherein the electrical stimulation current is delivered at a frequency less than about 100 Hz.

11. The method of claim 3 wherein the one or more drugs comprise at least one or more of an excitatory neurotransmitter, an excitatory neurotransmitter agonist, an inhibitory neurotransmitter antagonist, an agent that increases the level of an excitatory neurotransmitter, and an agent that decreases the level of an inhibitory neurotransmitter.

12. The method of claim 3 wherein the one or more drugs comprise at least one or more of glutamate and a glutamate receptor agonist.

13. The method of claim 3 further comprising:
applying the at least one stimulus in accordance with one or more stimulation parameters;
sensing at least one condition related to the movement disorder; and
using the at least one sensed condition to adjust the one or more stimulation parameters.

14. The method of claim 13 wherein the at least one sensed condition comprises at least one or more of an electrical activity of a muscular population, an electrical activity of a neural population, a neurotransmitter level, a change in a neurotransmitter level, a neurotransmitter breakdown product level, a change in a neurotransmitter breakdown product level, a medication level, a change in a medication level, a drug level, a change in a drug level, a hormone level, a change in a hormone level, an enzyme level, a change in an enzyme level, an interleukin level, a change in an interleukin level, a cytokine level, a change in a cytokine level, a lymphokine level, a change in a lymphokine level, a chemokine level, a change in a chemokine level, a growth factor level, a change in a growth factor level, a level of a bloodborne substance, a change in level of a bloodborne substance, a level of a substance in an interstitial fluid, a change in level of a substance in the interstitial fluid, a substance in a cerebrospinal fluid, and a change in a level of a substance in the cerebrospinal fluid.

15. The method of claim 3 further comprising implanting the at least one system control unit in the head and outside the patient's brain.

16. The method of claim 3 further comprising implanting the at least one system control unit entirely within an extradural area under the patient's skull.

17. The method of claim 3 further comprising implanting the at least one system control unit entirely within the patient's skull.

18. The method of claim 3 further comprising implanting the at least one system control unit subcutaneously beneath the patient's scalp and above the patient's skull.

19. The method of claim 3 further comprising implanting the at least one system control unit entirely in the patient's skull and an extradural area under the skull.

20. The method of claim 3 wherein the at least one system control unit conforms to a profile of the patient's skull.

21. A method of treating a patient with a movement disorder, comprising:
implanting in the patient's head at least one leadless system control unit for controlling delivery of at least one stimulus comprising an electrical stimulation current, said system control unit having an outer casing and a number of electrodes disposed on the casing;
applying the at least one stimulus via said electrodes to at least one area of the brain affecting a movement disorder in order to at least in part alleviate the movement disorder of the patient being treated;
selecting the at least one area of the brain from at least one of a motor cortex, a premotor cortex, and a sensory cortex; and
applying at least one inhibitory stimulus to the at least one selected area of the brain, thereby decreasing activity of the at least one selected area.

22. (canceled)

23. The method of claim 21 wherein the electrical stimulation current has a frequency greater than or equal to about 100 Hz.

24. The method of treatment of claim 21 wherein the system control unit is connected to at least one infusion outlet, and wherein the stimulus comprises stimulation via one or more drugs delivered through the at least one outlet.

25. The method of claim 24 wherein the one or more drugs comprise at least one or more of an excitatory neurotransmitter antagonist, an inhibitory neurotransmitter, an inhibitory neurotranrsmitter agonist, an agent that increases a level of an inhibitory neurotransmitter, an agent that decreases a level of an excitatory neurotransmitter, a local anesthetic agent, and an analgesic medication.

26. The method of claim 24 wherein the one or more drugs comprise at least one or more of dopamine, GABA, and a GABA agonist.

27. (canceled)

28. The method of claim 21 further comprising:
applying the at least one stimulus in accordance with one or more stimulation parameters;
sensing at least one condition related to the movement disorder; and
using the at least one sensed condition to adjust the one or more stimulation parameters.

29. The method of claim 28 wherein the at least one sensed condition comprises at least one or more of an electrical activity of a muscular population, an electrical activity of a neural population, a neurotransmitter level, a change in a neurotransmitter level, a neurotransmitter breakdown product level, a change in a neurotransmitter breakdown product level, a medication level, a change in a medication level, a drug level, a change in a drug level, a hormone level, a change in a hormone level, an enzyme level, a change in an enzyme level, an interleukin level, a change in an interleukin level, a cytokine level, a change in a cytokine level, a lymphokine level, a change in a lymphokine level, a chemokine level, a change in a chemokine level, a growth factor level, a change in a growth factor level, a level of a bloodborne substance, a change in level of a bloodborne substance, a level of a substance in an interstitial fluid, a change in level of a substance in the interstitial fluid, a substance in a cerebrospinal fluid, and a change in a level of a substance in the cerebrospinal fluid.

30. The method of claim 21 further comprising implanting the at least one system control unit in the head and outside the patient's brain.

31. The method of claim 21 further comprising imnplanting the at least one system control unit entirely within an extradural area under the patient's skull.

32. The method of claim 21 further comprising implanting the at least one system control unit entirely within the patient's skull.

33. The method of claim 21 further comprising implanting the at least one system control unit subcutaneously beneath the patient's scalp and above the patient's skull.

34. The method of claim 21 further comprising implanting the at least one system control unit entirely in the patient's skull and an extradural area under the patient's skull.

35. The method of claim 21 wherein the at least one system control unit conforms to a profile of the patient's skull.

36. (canceled)

37. A system for treating a patient with a movement disorder, said system comprising:
a leadless system control unit having an outer casing and a number of electrodes built into the casing; and
at least one infusion outlet coupled to said system control unit;
wherein said system control unit is configured to deliver an electrical stimulation current via said electrodes and one or more drugs via said at least one infusion outlet to an area within the brain of a patient in accordance with one or more stimulation parameters configured to treat said movement disorder; and
wherein said system control unit is further configured to be implanted within a head of said patient so as to deliver said electrical stimulation current and said one or more drugs to said area within said brain which comprises at least one or more of a motor cortex, premotor cortex, and sensory cortex.

38. The system of claim 37, further comprising:
a sensor for sensing at least one condition related to said movement disorder;
wherein said system control unit uses said at least one sensed condition to adjust one or more of said stimulation parameters.

39. The system of claim 38, wherein said at least one condition related to said movement disorder comprises at least one or more of an electrical activity of a muscular population, an electrical activity of a neural population, a neurotransmitter level, a change in a neurotransmitter level, a neurotransmitter breakdown product level, a change in a neurotransmitter breakdown product level a medication level, a change in a medication level, a drug level, a change in a drug level, a hormone level, a change in a hormone level, an enzyme level, a change in an enzyme level, an interleukin level, change in an interleukin level, a cytokine level, change in a cytokine level, a lymphokine level, a change in a lymphokine level, a chemokine level, a change in a chemokine level, a growth factor level, a change in a growth factor level, a level of a bloodborne substance, a change in level of a bloodborne substance, a level of a substance in an interstitial fluid, a change in level of a substance in the interstitial fluid, a substance in a cerebrospinal fluid, and a change in the level of a substance in the cerebrospinal fluid.
